(19) United States
(12) Patent Application Publication
(10) Pub. No.: US 2024/0382517 A1
(43) Pub. Date: Nov. 21, 2024

(54) Topical Pharmaceutical Skin Composition for the Delivery of Nitric Oxide in Combination with Other Topical Dermatologic Agents

(71) Applicant: NOxy Health Products, Inc., San Mateo, CA (US)

(72) Inventors: C. Michael MILLER, Anaconda, MT (US); Lex SCHINDLER, Denton, TX (US); David A. Bell, Farmington, UT (US); Tim Jacobson, San Mateo, CA (US)

(21) Appl. No.: 18/667,467

(22) Filed: May 17, 2024

Related U.S. Application Data
(60) Provisional application No. 63/503,319, filed on May 19, 2023.

(51) Int. Cl. A61K 33/00; A61K 9/00; A61K 9/12; A61K 31/167; A61K 36/61; A61K 36/886; A61K 47/12; A61K 47/18; A61K 47/22; A61P 17/02

(52) U.S. Cl. CPC A61K 33/00; A61K 9/0014; A61K 9/122; A61K 31/167; A61K 36/61; A61K 36/886; A61K 47/12; A61K 47/186; A61K 47/22; A61P 17/02

(57) ABSTRACT

Disclosed is a topical dermatological composition that generates nitric acid in situ for the treatment of dermatological conditions.

### BACKGROUND

A number of benefits have been identified regarding the use of nitric oxide (NO) to help or treat humans and animals having a variety of medical conditions, particularly those associated with the skin. Since nitric oxide is a gas and is considered an unstable molecule, it remains a challenge to develop dermatological topical agents that can consistently promote the formation of, and provide nitric oxide to, the skin of a human or animal. NO exerts a positive effect on skin health by providing immunomodulatory, vasodilatory, and antimicrobial benefits. Topical NO compositions are therefore being investigated to treat a range of skin conditions and pathologies ranging from minor skin cuts, burns, and abrasions, to diabetic foot ulcers, to other conditions such as acne and onychomycosis. Importantly, it has been found that bacteria do not become resistant to NO, and the concept of using NO in conjunction with other topical dermatologic agents, potentially in the same dosage form is an attractive goal to formulators. However, since an effective method of delivering NO is to generate it in situ, the chemical interaction between NO precursors and other topical dermatologic agents as well as excipients used in topical dermatologic compositions is a significant concern, as is NO stability itself in the resulting topical dermatologic composition. Maintaining the rate of NO generation as well as protecting generated NO from side reactions is one of the challenges formulators face in developing new combination NO/topical dermatologic agent products for the treatment of skin conditions and pathologies.

As a result, a challenging need remains for the development of effective combination NO/topical pharmaceutical agent compositions that can both dependably generate nitric oxide in situ and maintain the integrity of the topical dermatologic agent for treatment viability.

### SUMMARY

These and other needs are met by the present invention which provides a topical dermatological composition comprising:
a first aqueous solution comprising:
   1-20 by weight percent of at least one protic acid based on the total weight of the first solution;
   0.1 to 10 by weight percent of a surfactant based on the total weight of the first solution;
a second aqueous solution comprising:
   1-35 by weight percent of a nitric oxide precursor based on the total weight of the second solution;
   0.1 to 10 by weight percent of a surfactant based on the total weight of the second solution;
wherein at least one or both of the first and second solutions contain one or more additional topical dermatologic agents; and
wherein a resultant mixed medium containing nitric oxide is produced when the first and second solutions are mixed.

In another aspect, what is provided is a topical dermatological composition comprising:
a first aqueous solution comprising:
   1-20 by weight percent of at least one protic acid based on the total weight of the first solution;
a second aqueous solution comprising:
   1-35 by weight percent of a nitric oxide precursor based on the total weight of the second solution;
wherein at least one or both of the first and second solutions contain one or more additional topical dermatologic agents; and
wherein a resultant mixed medium containing nitric oxide is produced when the first and second solutions are mixed.

In another aspect, what is provided is a pharmaceutical composition for topical administration comprising: water, nitric oxide, at least one additional topical dermatologic agent, and optionally a surfactant.

In another aspect, what is provided is a pharmaceutical composition for topical administration comprising: water, nitric oxide, at least one additional topical dermatologic agent, and optionally a surfactant.

In another aspect, what is provided is a pharmaceutical composition for topical administration comprising: water, nitric oxide, at least one additional topical dermatologic agent, and optionally a surfactant, wherein the pharmaceutical composition is slightly acidic or neutral to minimize skin irritation.

The pharmaceutical composition for topical administration is readily applied as a liquid, ointment, cream, foam, gel, slurry, liquid-liquid emulsion, lotion, or paste, that delivers nitric oxide to the skin. For example, the pharmaceutical composition for topical administration can be applied to a cut, burn, or scratch, or for another skin condition such as eczema, acne, psoriasis, rosacea, wounds, and the like, for a period of time so that the nitric oxide and the additional topical dermatologic agent remains in contact with the skin site for a sufficient time to provide treatment.

We have found that it is possible to prepare a topical dermatologic composition for the in situ generation of nitric oxide that contains other topical dermatologic agents as well as excipients used in topical dermatologic compositions without deleteriously affecting the generation of nitric oxide. The topical dermatologic compositions as disclosed herein incorporate other therapeutically effective topical dermatologic agents without affecting the quality or deliverability of the nitric oxide containing dermatologic composition, preserving the ability of the composition to deliver nitric oxide to a location on the skin that requires treatment, while maintaining or improving therapeutic efficacy of the nitric oxide due to the presence of the additional topical dermatologic agent.

### BRIEF DESCRIPTION OF THE FIGURES

FIG. 1 depicts the NOx product profiles for three topical compositions containing benzalkonium chloride.

FIG. 2 depicts the average product profile from the three topical compositions containing benzalkonium chloride in FIG. 1 as compared to a sample that does not contain benzalkonium chloride.

FIG. 3 depicts NOx production from a topical NO/salicylic acid composition.

FIG. 4 depicts NO production from a topical NO/ascorbic acid composition.

FIG. 5 depicts NO production from a topical NO/buffered hypochlorous acid composition.

FIG. 6 depicts NOx production from topical NO/lidocaine compositions.

FIG. 7 depicts NOx production from topical NO/lidocaine compositions.

FIG. 8 depicts NOx production from a topical NO/Manuka honey composition.

### DETAILED DESCRIPTION

### Definitions

As used herein, an aqueous solution is a solution in which water is present, alone or in combination, with another pharmaceutically acceptable liquid which is miscible with water or another pharmaceutically acceptable solid that is soluble in the aqueous solution.

As used herein, “nitric oxide precursor” refers to a chemical source of nitric oxide, including nitrates or nitrites, such as sodium nitrite or potassium nitrite. Other sources of nitric oxide precursors include diazeniumdiolates (NONOates), L-arginine, L-citrulline, S-nitrosothiols, nitrosamines, nitroglycerin, nitroprusside, and NO-metal complexes, among others.

As used herein, coco betaine and cocamidopropyl betaine are different compounds and are examples of cationic biobased surfactants produced from natural raw materials such as coconut oil.

As used herein, “dermatological agent” and “dermatological topical agent” can be used interchangeably and refer to therapeutic agents as well as topical excipients that can be applied directly on the skin to treat skin conditions. They deliver medicines to treat or prevent dermatologic conditions or are used as excipients for topical dermatologic products or in wound dressings. As is known to the skilled person, many topical dermatologic agents and excipients described herein may serve more than one purpose, and it is not intended for the topical dermatologic agents and excipients to be limited to a single purpose.

Types of dermatological agents include topical agents for miscellaneous use, topical acne agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacients, topical steroids, topical steroids with anti-infectives, topical cleansers, topical steroids with anti-infectives, ingredients that help in angiogenesis, anorectal agents, skin protectants, odor managers, as well as moisture managers, topical enzymes, ingredients in wound dressings, and topical antiseptics. Topical excipients include humectants, emulsifiers, preservatives, adhesives, thickeners, and others as described herein.

Dermatologic agents are used to treat a variety of skin conditions and may have multiple biological effects. Non-limiting examples of topical dermatologic agents used to treat skin conditions that are described herein include aluminum chloride hexahydrate, crisaborole (CAS Reg. No. 906673-24-3), eflornithine, tacrolimus, pimecrolimus, minoxidil, glycopyrronium, menthol, ionic zinc, coal tar, capsaicin, selenium sulfide, bimatoprost, diphenhydramine, sodium hyaluronate, salicylic acid, Vitamin E, bexarotene, mequinol/tretinoin, becaplermin, dexpanthenol, and alitretinoin.

Topical acne agents include antiseptic washes that contain ingredients to gently cleanse the skin; and creams, lotions, or gels that exfoliate the skin, inhibit bacterial growth, speed up skin cell renewal or decrease the formation of comedones. Non-limiting examples of topical acne agents include adapalene, benzoyl peroxide, clindamycin, dapsone, tretinoin, azelaic acid, tazarotene, salicylic acid, clascoterone, erythromycin, and resorcinol.

Topical anesthetics are medicines that numb and reduce the sensation of pain in the area to which they are applied. Non-limiting examples of topical anesthetics include lidocaine, pramoxine, phenol, benzocaine, dibucaine, xylocaine, tetracaine, prilocaine, benzocaine, wintergreen (CAS Reg. No. 68917-75-9), and dyclonine.

Topical anti-infective agents act by either killing or inhibiting the spread of infectious agents. They include antibiotics, antibacterial (antimicrobial), antifungal and antiviral agents. Non-limiting examples of topical anti-infectives include docosanol, ivermectin, imiquimod, hydrogen peroxide, crotamiton, spinosad, cadexomer iodine, malathion, piperonyl butoxide, pyrethrins, permethrin, sinecatechins, abametapir, acetic acid, iodoquinol, nitrofurazone, and chloroxine, mupirocin (CAS Reg. No. 12650-69-0), polyhexanide (polyaminopropyl biguanide), crystal violet (CAS Reg. No. 548-62-9), gentian violet (CAS Reg. No. 548-62-9), lemon, sodium hypochlorite, and povidone-iodine solution, polymyxin (CAS Reg. No. 1405-20-5), carvacrol (CAS Reg. No. 499-75-2), sandalwood oil (CAS Reg. No. 8006-87-9), thymol (CAS Reg. No. 89-83-8), decanoic acid, Methylene blue (CAS Reg. No. 61-73-4), hypochlorous acid, sorbic acid, acetic acid, grapefruit extract (CAS 90045-43-5), and Medicinal honey.

Topical anti-rosacea agents are used for the treatment of inflammatory papules, pustules and erythema of rosacea. Non-limiting examples of anti-rosacea agents include ivermectin, brimonidine, oxymetazoline, and azelaic acid.

Topical antibiotics are medicines that destroy or inhibit the growth of susceptible bacteria. Non-limiting examples of topical antibiotics include sulfacetamide sodium/sulfur, retapamulin, ozenoxacin, erythromycin, polymyxin b, mafenide, gentamicin, and mupirocin.

Topical antifungals are products that treat fungal infections caused by dermatophytes, yeasts, or mold. Non-limiting examples of topical antifungals include efinaconazole, tavaborole, ketoconazole, terbinafine, undecylenic acid, nystatin, econazole, ciclopirox, naftifine, oxiconazole, clotrimazole, sertaconazole, tolnaftate, butenafine, luliconazole, miconazole, and sulconazole.

Topical antihistamines are products that have been manufactured for use on the skin, in the nose, or in the eye. They contain antihistamines which are medicines that block histamine release from histamine-1 receptors and are used to treat the symptoms of an allergic reaction such as edema (swelling), itch, inflammation (redness), sneezing, or a runny nose or watery eye. Non-limiting examples of topical antihistamines include doxepin and diphenhydramine.

Topical antineoplastics work by different mechanisms to prevent the development and spread of neoplastic cells that characterize cancers such as melanoma. Non-limiting examples of topical antineoplastics include imiquimod, fluorouracil, ingenol, ruxolitinib, tirbanibulin, and mechlorethamine.

Topical antipsoriatics are agents, which are applied on the skin surface to treat psoriasis. Non-limiting examples of topical antipsoriatics include betamethasone, calcipotriene, tazarotene, and halobetasol.

Topical antiviral agents are applied locally to treat viral infections. Non-limiting examples of topical antivirals include penciclovir and acyclovir.

Topical astringents are agents that cause skin cells or mucus membranes to contract or shrink, by precipitating proteins from their surface. When applied topically they dry, harden and protect the skin. A non-limiting example of a topical astringent is witch hazel.

Topical debriding agents are chemicals that are used locally to clean an open wound by removing foreign material and dead tissue, so that the wound heals without increased risk of infection. This makes the healing faster. Non-limiting examples of topical debriding agents include peru balsam (CAS Reg. No. 8007-00-9) combined with castor oil and trypsin, and anacaulase.

Topical depigmenting agents work in different ways to inhibit melanogenesis (the pigmentation pathway by which cells produce melanin). Some agents cause reversible depigmentation and some cause irreversible depigmentation. These agents are applied on the skin, on the affected area to treat hyperpigmentation. Non-limiting examples of topical depigmenting agents include fluocinolone combined with hydroquinone and tretinoin, and monobenzoin.

Topical emollients, or moisturizers, contain ingredients that soothe and soften the skin. Non-limiting examples of topical emollients include salicylic acid/urea, ammonium lactate, urea, petrolatum, lanolin, mineral oil, cetyl dimethicone copolyol, cyclomethicone, palm glyceride(s), panthenol (CAS Reg. No. 81-13-0), and aloe vera.

Topical keratolytics are agents that are applied on the skin to soften the keratin. They loosen and assist exfoliation of the skin cells. Keratolytics also help the skin to bind moisture and are useful in treating dry skin conditions. They are used to treat psoriasis, acne, warts, corns and other forms of keratosis. Non-limiting examples of topical keratolytics include podofilox (CAS Reg. No. 518-28-5), salicylic acid, hydrocolloids, and trichloroacetic acid.

Topical non-steroidal anti-inflammatories (often abbreviated to NSAIDs) are creams, gels, rubs, solutions or sprays that contain a nonsteroidal anti-inflammatory agent and are designed to be applied directly to the skin overlying a painful joint or area of bone. They are used to relieve pain and to treat symptoms of arthritis such as inflammation, swelling, and stiffness. Topical NSAIDs may also be used in the treatment of actinic keratosis (a precancerous patch of thick, scaly or crusted skin). Non-limiting examples of topical NSAIDs include diclofenac, capsaicin/diclofenac, and diclofenac/lidocaine.

Topical photochemotherapeutics make skin more sensitive to light. They work by causing a reaction with light that can destroy certain types of diseased skin cells. They may be used in the treatment of vitiligo or actinic keratosis in combination with light treatment. Non-limiting examples of topical photochemotherapeutics include aminolevulinic acid, methoxsalen, methylamino levulinate, and aminolevulinic acid.

Topical rubefacients cause irritation and reddening of the skin, due to increased blood flow. They are used in the treatment of pain in various musculoskeletal conditions. Non-limiting examples of topical rubefacients include methyl salicylate, camphor, menthol, and capsaicin/menthol.

Topical steroids contain corticosteroids (often abbreviated to steroids) which are designed to be applied externally to the scalp or the skin, depending on the condition being treated. Corticosteroids control inflammation by mimicking naturally occurring corticosteroid hormones produced by adrenal glands, which are two small glands that sit on top of kidneys. In addition to reducing inflammation (redness and swelling) in the area that they are applied, topical corticosteroids also suppress the immune response, reduce cell turnover, and constrict (narrow) blood vessels. Non-limiting examples of topical steroids include mometasone, clobetasol, triamcinolone, fluocinonide, flurandrenolide, clocortolone, halobetasol, desoximetasone, desonide, betamethasone, halcinonide, fluocinolone, prednicarbate, diflorasone, triamcinolone, fluticasone, and alclometasone.

Topical antiseptics are used to reduce the microbial count and reduce the risk of infections on the skin. Non-limiting examples of topical antiseptics include alcohol (ethanol and isopropanol), benzethonium chloride, benzalkonium chloride (BAC), camphorated metacresol, eucalyptol 0.091%, hexylresorcinol, hydrogen peroxide topical solution, iodine tincture, iodine topical solution, menthol, methylbenzethonium chloride, methyl salicylate, phenol, povidone-iodine, menthol, citric acid, sodium tetraborate, benzoic acid, polyaminopropyl biguanide, polyhexylmethylene biguanide, and thymol. BAC represents a mixture of N,N-dimethyl alkyl amines, which conform generally to the formula:

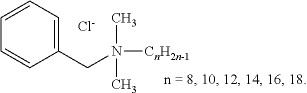

“Topical dermatologic agent” also includes agents that can aid topically in angiogenesis such as trans-cinnamaldehyde (CAS Reg. No. 14371-10-9); and anorectal agents such as oak extract (CAS Reg. No. 68917-11-3).

“Topical dermatologic agent” also includes skin protectants such as listed in the following table.

| Skin Protectant | CAS Reg. No. |
|---|---|
| Allantoin | 97-59-6 |
| Aluminum sulfate | 10043-0103 |
| Octadecahydrate | 7784-31-8 |
| Lanolin (hydrous) | 8006-54-0 |
| Petrolatum (petroleum jelly) | 8009-03-8 |
| Konjac flour | 37220-17-0 |
| Isopropyl sorbate | 55584-26-4 |
| Keratin | 68238-35-7 |
| Palmitamide MEA | 629-54-9 |
| Zirconium oxide | 1314-23-4 |
| Sucrose octasulfate/ | 54182-58-0 |
| Aluminum hydrochloride | |
| Glycerin | 56-81-5 |
| Ascorbic acid | 50-81-7 |
| Fruit extract | |
| Glycrrhetinic acid | 471-53-4 |
| (Licorice extract) | |
| Lavender | 8000-28-0 |
| Meadowsweet extract | 7732-18-5 |
| | 56-81-5 |
| | 84775-57-5 |
| | 122-99-6 |
| Myrtillus extract | 84082-34-8 |
| Oat glucan | 9041-22-9 |
| | 9051-97-2 |
| Soy protein | 9010-10-0 |
| Triglycerol | 20411-31-8 |

“Topical dermatologic agent” also includes adhesives that can be used to attach wound dressings to the skin. Examples include hydrogenated rosin (CAS Reg. No. 64365-17-9).

“Topical dermatologic agent” also includes topical cleansers such as aqueous saline, and cocoamphodiacetate (CAS Reg. No. 68650-39-5).

“Topical dermatologic agent” also includes humectants, which are substances that attract water, that can be used in wound dressings and include such substances as butylene glycol, propylene glycol, betaines, ethoxydiglycol, pentylene glycol, acetamide MEA (CAS Reg. No. 142-26-7), lactic acid, and sodium lactate.

“Topical dermatologic agent” also includes substances for odor management in wound dressings such as sodium copper chlorophyllin (CAS Reg. No. 65963-40-8).

“Topical dermatologic agent” also includes preservatives such as diazolidinyl urea, DMDM hydantoin (CAS Reg. No. 6440-58-0), hydrochloric acid, maltodextrin, phenoxyethanol, phosphoric acid, potassium sorbate, sodium benzoate, sodium citrate, sodium metabisulfite, sodium sulfate, Germaben® II (CAS Reg. No. 57-55-6, 78491-02-8), and Quaternium-15 (CAS Reg. 4080-31-3, 51229-78-8 (cis isomer)).

“Topical dermatologic agent” also includes topical emulsifiers such as sucrose laurate, tartaric acid, triethanolamine, Tara gum (CAS Reg. No. 39300-88-4), ceteareth-10 phosphate (CAS Reg. No. 106233-09-4), triethanolamine salicylate, and diethanolamine cetyl phosphate.

Topical thickening agents include xanthan gum, among others.

“Topical dermatologic agent” also includes agents for moisture management in wound dressings such as calcium carbonate, calcium alginate, gelling fiber from chitosan, polyvinyl alcohol fibers, carboxymethyl cellulose fibers, and hydrocolloids.

“Topical dermatologic agent” also includes topical enzymes that can be used topically, such as glucose oxidase, anacaulase, and lactoperoxidase.

Miscellaneous dermatologic agents include manganese chloride, magnesium sulfate, wheat extract, bisabolol (chamomile oil, CAS Reg. No. 23089-26-1), Camellia sinensis (leaf extract, CAS Reg. No. 84650-60-2), Centella asiatic extract (CAS Reg. No. 84696-21-9), blueberry extract (CAS Reg. No. 84082-34-8), grape seed extract (CAS Reg. No. 84929-27-1), sarcosine, Ringer's lactate (CAS Reg. No. 8022-63-7), piroctone oleamine (CAS Reg. No. 68890-66-4), sulfur dioxide, honey including Manuka honey, hydrogels, and temesteine, recombinant human platelet derived growth factor, Karaya gum (cas Reg. No. 9000-36-6), carboxymethyl cellulose, silicone gels containing polydimethylsiloxane, hydroxyl- and dihydroxysiloxane, vitamin a, d, and e, ascorbyl tetraisopalmitate, and alkyl siloxane resin. Miscellaneous dermatological agents also include ingredients in wound dressings such as ferric chloride hexahydrate, calcium salts, thrombin, potassium ferrate, and RADA-16 peptide.

All amounts are given as weight percents based on the total weight (i.e., 100 weight percent) of a given solution.

As used herein, “therapeutic effect” means treatment of any kind, the results of which are judged to be useful or favorable

### Topical Pharmaceutical Composition Embodiments

As provided above, in one aspect, what is provided is a topical dermatological composition, comprising:
a first aqueous solution comprising:
   1-20 weight percent of at least one protic acid based on the total weight of the first solution;
   0.1 to 10 weight percent of a surfactant based on the total weight of the first solution;
a second aqueous solution comprising:
   1-35 weight percent of a nitric oxide precursor based on the total weight of the second solution;
   0.1 to 10 weight percent of a surfactant based on the total weight of the second solution;
wherein at least one or both of the first and second solutions contain one or more additional topical dermatologic agents; and
wherein a resultant mixed medium containing nitric oxide is produced when the first and second solutions are mixed.

In another aspect, what is provided is a topical dermatological composition comprising:
a first aqueous solution comprising:
   1-20 by weight percent of at least one protic acid based on the total weight of the first solution;
a second aqueous solution comprising:
   1-35 by weight percent of a nitric oxide precursor based on the total weight of the second solution;
wherein at least one or both of the first and second solutions contain one or more additional topical dermatologic agents; and
wherein a resultant mixed medium containing nitric oxide is produced when the first and second solutions are mixed.

### Components

### Protic Acid

In one embodiment, the at least one protic acid typically has a pH of 1.5 to a pH of 6. In another embodiment, the at least one protic acid is selected from the group consisting of citric acid, lactic acid, salicylic acid, phosphoric acid, ascorbic acid, hydrochloric acid, acetic acid, hyaluronic acid, hypochlorous acid, gluconic acid, aspartic acid, formic acid, fumaric acid, galacturonic acid, malonic acid, formic acid, acetoglutaric acid, gluconic acid, glutamic acid, butyric acid, glutaric, butyric acid, shikimic acid, propionic acid, pyruvic glyoxylic acid, 2-hydroxybutyric acid, α-hydroxyglutaric acid, isocitric acid, lactic acid, malic acid, methylmalonic acid, quinic acid, succinic acid, tartaric acid, and oxalic acid, or a combination thereof. Honey, including Manuka honey, acacia honey, clover honey, orange blossom honey, various wildflower honeys, as well as a variety of other honeys is the source for a combination of protic acids, including gluconic acid, aspartic acid, citric, acetic, formic, fumaric, galacturonic, malonic, formic, acetoglutaric, gluconic, glutamic, butyric, glutaric, butyric, shikimic, propionic, pyruvic glyoxylic, 2-hydroxybutyric, α-hydroxyglutaric, isocitric, lactic, malic, methylmalonic, quinic, succinic, tartaric, oxalic and others.

In a further embodiment, the at least one protic acid is selected from the group consisting of citric acid, ascorbic acid, hypochlorous acid, and honey, or a combination thereof.

In a further embodiment, the at least one protic acid is Manuka honey. Manuka honey is a honey that is produced from the nectar of the Manuka tree, Leptospermum scoparium that has antibacterial and anti-inflammatory qualities that make it useful for treating a variety of skin conditions.

In a further embodiment, the at least one protic acid is citric acid.

### Surfactants

In one embodiment, the surfactant is a pharmaceutically acceptable cationic surfactant that can be used topically on the skin. In a further embodiment, the surfactants are selected from cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride.

In one embodiment, the surfactant is cocamidopropyl betaine.

In a further embodiment, the surfactant is coco betaine.

### Nitric Oxide Precursor

In one embodiment, the nitric oxide precursor is a nitrite salt that is compatible with human and/or animal physiology for transdermal therapeutic use.

In one embodiment, the nitric oxide precursor is selected from the group consisting of nitrates or nitrites, such as sodium nitrite or potassium nitrite diazeniumdiolates (NONOates), L-arginine, L-citrulline, S-nitrosothiols, nitrosamines, nitroglycerin, nitroprusside, and NO-metal complexes. In one embodiment the nitrite salt is selected from the group consisting of sodium nitrite, calcium nitrite, potassium nitrite, and ammonium nitrite. In another embodiment, the nitric oxide precursor is sodium nitrite or potassium nitrite.

In one embodiment, the nitric oxide precursor is sodium nitrite.

### Topical Dermatologic Agent

In one embodiment, at least one of the solutions of the topical dermatologic composition further comprises a topical dermatologic agent or a combination of dermatologic agents.

In another embodiment, both solutions comprise at least one topical dermatologic agent or combination of agents.

In another embodiment, the first solution comprises at least one topical dermatologic agent, and the second solution comprises a different topical dermatologic agent.

In these and other embodiments, the topical dermatologic agent is selected from the agents described herein. In these and other embodiments, the topical dermatologic agent is a therapeutic dermatologic agent or excipients used in wound dressings selected from the group consisting of agents to treat acne, topical anesthetics, topical anti-infectives including antibiotic, antimicrobials, and antivirals agents, topical anti-rosacea agents, topical antifungal agents, topical antihistamines, topical astringents, topical debriding agents, topical depigmenting agents, topical emollients, topical keratolytics, topical non-steroidal anti-inflammatory agents, topical rubefacient agents, topical steroid agents, topical antiseptic agents, topical agents that aid in angiogenesis, topical skin protectants, adhesives, topical skin cleansers, topical humectants, odor management agents as well as perfumes and fragrance oils, preservatives, emulsifiers, agents for moisture management, enzymes that can be used topically, excipients that can be used in topical skin compositions as well as wound dressings, as well as antineoplastics, antipsoriatics, photochemotherapeutics, topical steroids with anti-infectives, anorectal and thickeners.

In one embodiment, the topical dermatologic agent is selected from the group consisting of topical acne agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical, depigmenting agents, topical emollients, topical keratolytics, topical non-steroidal anti-inflammatories, topical photochemotherapeutics, topical rubefacients, topical steroids, topical steroids with anti-infectives, and topical antiseptics, or a combination thereof. Examples of various topical dermatologic agents are provided in the definitions section of this application.

In another embodiment, the topical dermatologic composition disclosed herein comprises a topical dermatologic antiseptic for first aid use to treat wounds including scrapes, cuts and burns. In this and other embodiments, the topical dermatological antiseptic is selected from the group consisting of alcohol (ethanol and isopropanol), benzethonium chloride, benzalkonium chloride (BAC), camphorated metacresol, eucalyptol 0.091%, hexylresorcinol, hydrogen peroxide topical solution, iodine tincture, iodine topical solution, menthol, methylbenzethonium chloride, methyl salicylate, phenol, povidone-iodine, menthol, citric acid, sodium tetraborate, benzoic acid, polyaminopropyl biguanide, polyhexylmethylene biguanide, and thymol. BAC represents a mixture of N,N-dimethyl alkyl amines, which conform generally to the formula:

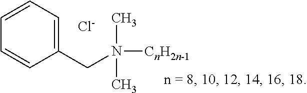

In another embodiments, the topical dermatologic antiseptic is selected from the group consisting of ethanol, isopropanol, benzethonium chloride, benzalkonium chloride (BAC), camphorated metacresol, eucalyptol 0.091%, hexylresorcinol hydrogen peroxide topical solution, iodine tincture, iodine topical solution, menthol, hypochlorous acid, methylbenzethonium chloride, methyl salicylate, phenol, povidone-iodine, and thymol.

In another embodiment, two or more topical dermatologic agents are present in the first or second solutions. In one embodiment, one topical dermatologic agent is present in one of the solutions and another topical dermatologic agent is present in the other solution. In another embodiment, both topical dermatologic agents are present in one of the solutions and the other solution does not include one or both of the topical dermatologic agents. In another embodiment, both topical dermatologic agents are present in both solutions.

In another embodiment, three or more topical dermatologic agents are present in the first or second solutions. In one embodiment, one topical dermatologic agent is present in one solution, and the other two topical dermatologic agents are present in the other solution. In another embodiment, all three topical dermatologic agents are present in one of the solutions and the other solution does not include the topical dermatologic agents. In another embodiment, all three topical dermatologic agents are present in both solutions.

In these and other embodiments, the first, second, and third topical dermatologic agents are selected from the group consisting of topical acne agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacients, topical steroids, topical steroids with anti-infectives, and topical antiseptics, or a combination thereof. Examples of various topical dermatologic agents are provided in the definitions section of this application.

In these and the embodiments, the topical dermatologic agent is present based on the total weight of either the first solution, second solution, or both the first and second solutions, of 0.01 to 5 weight percent.

### First Solution

In one embodiment, the first solution is an aqueous solution comprising:
1 to 20 percent by weight of the at least one protic acid based on the total weight of the first solution;
0.1 to 10 percent by weight of a surfactant based on the total weight of the first solution; and
0.05 to 0.2 percent by weight of an additional topical dermatologic agent based on the total weight of the first solution.

In this and other embodiments, the surfactant is selected from the group consisting of cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride

In another embodiment, the first solution is an aqueous solution comprising:
1 to 20 percent by weight of the at least one protic acid based on the total weight of the first solution;
0.1 to 10 percent by weight of cocamidopropyl betaine or coco-betaine based on the total weight of the first solution; and
0.05 to 0.2 percent by weight of an additional topical dermatologic agent based on the total weight of the first solution.

In a further embodiment, the first solution is an aqueous solution comprising:
3 to 18 percent by weight of the at least one protic acid based on the total weight of the first solution;
0.5 to 8 percent by weight of cocamidopropyl betaine or coco-betaine based on the total weight of the first solution; and
0.08 to 0.18 percent by weight of an additional topical dermatologic agent based on the total weight of the first solution.

In a further embodiment, the first solution is an aqueous solution comprising:
5 to 15 percent by weight of the at least one protic acid based on the total weight of the first solution;
1 to 5 percent by weight of cocamidopropyl betaine or coco-betaine based on the total weight of the first solution; and
0.1 to 0.16 percent by weight of an additional topical dermatologic agent based on the total weight of the first solution.

In a further embodiment, the first solution is an aqueous solution comprising:
8 to 12 percent by weight of the at least one protic acid based on the total weight of the first solution;
2 to 4 percent by weight of cocamidopropyl betaine or coco-betaine based on the total weight of the first solution; and
0.11 to 0.15 percent by weight of an additional topical dermatologic agent based on the total weight of the first solution.

In one embodiment, the first solution is an aqueous solution comprising:
1 to 20 percent by weight of the at least one protic acid based on the total weight of the first solution;
0.1 to 10 percent by weight of cocamidopropyl betaine or coco-betaine based on the total weight of the first solution; and
0.05 to 0.2 percent by weight of an antiseptic agent based on the total weight of the first solution.

In a further embodiment, the first solution is an aqueous solution comprising:
3 to 18 percent by weight of the at least one protic acid based on the total weight of the first solution;
0.5 to 8 percent by weight of cocamidopropyl betaine or coco-betaine based on the total weight of the first solution; and
0.08 to 0.18 percent by weight of an antiseptic agent based on the total weight of the first solution.

In a further embodiment, the first solution is an aqueous solution comprising:
5 to 15 percent by weight of the at least one protic acid based on the total weight of the first solution;
1 to 5 percent by weight of cocamidopropyl betaine or coco-betaine betaine based on the total weight of the first solution; and
0.1 to 0.16 percent by weight of an antiseptic agent based on the total weight of the first solution.

In a further embodiment, the first solution is an aqueous solution comprising:
5 to 15 percent by weight of the at least one protic acid based on the total weight of the first solution;
1 to 5 percent by weight of cocamidopropyl betaine or coco-betaine based on the total weight of the first solution; and
0.11 to 0.15 percent by weight of an antiseptic agent based on the total weight of the first solution.

In another embodiment, the first solution is an aqueous solution comprising:
1 to 20 percent by weight of the at least one protic acid based on the total weight of the first solution;
0.1 to 10 percent by weight of cocamidopropyl betaine or coco-betaine based on the total weight of the first solution; and
0.05 to 0.2 percent by weight of BAC based on the total weight of the first solution.

In a further embodiment, the first solution is an aqueous solution comprising:
3 to 18 percent by weight of the at least one protic acid based on the total weight of the first solution;
0.5 to 8 percent by weight of cocamidopropyl betaine or coco-betaine based on the total weight of the first solution; and
0.08 to 0.18 percent by weight of BAC based on the total weight of the first solution.

In a further embodiment, the first solution is an aqueous solution comprising:
5 to 15 percent by weight of the at least one protic acid based on the total weight of the first solution;
1 to 5 percent by weight of cocamidopropyl betaine or coco-betaine based on the total weight of the first solution; and
0.1 to 0.16 percent by weight of BAC based on the total weight of the first solution.

In a further embodiment, the first solution is an aqueous solution comprising:
5 to 15 percent by weight of the at least one protic acid based on the total weight of the first solution;
1 to 5 percent by weight of cocamidopropyl betaine or coco-betaine based on the total weight of the first solution; and
0.11 to 0.15 percent by weight of BAC based on the total weight of the first solution.

### Second Solution

In one embodiment, the second solution is an aqueous solution comprising:
1-35 weight percent of a nitric oxide precursor based on the total weight of the second solution;
0.1 to 10 weight percent of cocamidopropyl betaine or coco-betaine based on the total weight of the second solution; and
0.05 to 0.2 weight percent of an additional topical dermatologic agent based on the total weight of the second solution.

In this and other embodiments, the surfactant is selected from the group consisting of cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride

In another embodiment, the second solution is an aqueous solution comprising:
1-35 weight percent of a nitric oxide precursor based on the total weight of the second solution;
0.1 to 10 weight percent of cocamidopropyl betaine or coco-betaine based on the total weight of the second solution; and
0.05 to 0.2 weight percent of an additional topical dermatologic agent based on the total weight of the second solution.

In another embodiment, the second solution is an aqueous solution comprising:
5-25 weight percent of a nitric oxide precursor based on the total weight of the second solution;
0.3 to 8 weight percent of cocamidopropyl betaine or coco-betaine based on the total weight of the second solution; and
0.08 to 0.18 weight percent of an additional topical dermatologic agent based on the total weight of the second solution.

In another embodiment, the second solution is an aqueous solution comprising:
10 to 20 weight percent of a nitric oxide precursor based on the total weight of the second solution;
0.5 to 5 weight percent of cocamidopropyl betaine or coco-betaine based on the total weight of the second solution; and
0.1 to 0.16 weight percent of an additional topical dermatologic agent based on the total weight of the second solution.

In another embodiment, the second solution is an aqueous solution comprising:
10 to 20 weight percent of a nitric oxide precursor based on the total weight of the second solution;
0.5 to 5 weight percent of cocamidopropyl betaine or coco-betaine based on the total weight of the second solution; and
0.11 to 0.15 weight percent of an additional topical dermatologic agent based on the total weight of the second solution.

In one embodiment, the second solution is an aqueous solution comprising:
1-35 weight percent of a nitric oxide precursor based on the total weight of the second solution;
0.1 to 10 weight percent of cocamidopropyl betaine or coco-betaine based on the total weight of the second solution; and
0.05 to 0.2 weight percent of an antiseptic agent based on the total weight of the second solution.

In another embodiment, the second solution is an aqueous solution comprising:
5-25 weight percent of a nitric oxide precursor based on the total weight of the second solution;
0.3 to 8 weight percent of cocamidopropyl betaine or coco-betaine based on the total weight of the second solution; and
0.08 to 0.18 weight percent of an antiseptic agent based on the total weight of the second solution.

In another embodiment, the second solution is an aqueous solution comprising:
10 to 20 weight percent of a nitric oxide precursor based on the total weight of the second solution;
0.5 to 5 weight percent of cocamidopropyl betaine or coco-betaine based on the total weight of the second solution; and
0.1 to 0.16 weight percent of an antiseptic agent based on the total weight of the second solution.

In another embodiment, the second solution is an aqueous solution comprising:
10 to 20 weight percent of a nitric oxide precursor based on the total weight of the second solution;
0.5 to 5 weight percent of cocamidopropyl betaine or coco-betaine based on the total weight of the second solution; and
0.11 to 0.15 weight percent of an additional topical dermatologic agent based on the total weight of the second solution.

In another embodiment, the second solution is an aqueous solution comprising:
1-35 weight percent of a nitric oxide precursor based on the total weight of the second solution;
0.1 to 10 weight percent of cocamidopropyl betaine or coco-betaine based on the total weight of the second solution; and
0.05 to 0.2 weight percent of BAC based on the total weight of the second solution.

In another embodiment, the second solution is an aqueous solution comprising:
5-25 weight percent of a nitric oxide precursor based on the total weight of the second solution;
0.3 to 8 weight percent of cocamidopropyl betaine or coco-betaine based on the total weight of the second solution; and
0.08 to 0.18 weight percent of BAC based on the total weight of the second solution.

In another embodiment, the second solution is an aqueous solution comprising:
10 to 20 weight percent of a nitric oxide precursor based on the total weight of the second solution;
0.0.5 to 5 weight percent of cocamidopropyl betaine or coco-betaine based on the total weight of the second solution; and
0.1 to 0.16 weight percent of BAC based on the total weight of the second solution.

In another embodiment, the second solution is an aqueous solution comprising:
10 to 20 weight percent of a nitric oxide precursor based on the total weight of the second solution;
0.5 to 5 weight percent of cocamidopropyl betaine or coco-betaine based on the total weight of the second solution; and
0.11 to 0.15 weight percent of an additional topical dermatologic agent based on the total weight of the second solution.

In another embodiment, the first and second solutions are as described in Table 1.

TABLE 1

| | Mass | Ingredient Conc. |
|---|---|---|
| | (g) | (w/w %) |
| First | | |
| Solution (A) | | |
| Formulation | | |
| Water | 38.26 | 78.52% |
| Surfactant | 4.72 | 9.68% |
| Acid | 4.48 | 9.20% |
| Antiseptic | | |
| Second | | |
| Solution (B) | | |
| Formulation | | |
| Water | 39.42 | 78.85% |
| Surfactant | 1.62 | 3.23% |
| Sodium | 7.66 | 15.32% |
| Nitrite | | |
| Antiseptic | | |

In this embodiment, the concentration of the antiseptic is in the range of 0.01 to 95 weight/weight percent, which includes the endpoints. In another embodiment, the concentration of the antiseptic is in the range of 0.05 to 20 weight/weight percent, which includes the endpoints. In another embodiment, the concentration of the antiseptic is in the range of 0.1 to 10 weight/weight percent, which includes the endpoints.

In another embodiment, the first and second solutions are as described in Table 2.

TABLE 2

| | Mass | Ingredient Conc. |
|---|---|---|
| | (g) | (w/w %) |
| First | | |
| Solution (A) | | |
| Formulation | | |
| Water | 38.26 | 78.52% |
| Surfactant | 4.72 | 9.68% |
| Acid | 4.48 | 9.20% |
| Antiseptic | | |
| Second | | |
| Solution (B) | | |
| Formulation | | |
| Water | 39.42 | 78.85% |
| Surfactant | 1.62 | 3.23% |
| Sodium | 7.66 | 15.32% |
| Nitrite | | |

In this embodiment, the concentration of the antiseptic is in the range of 0.01 to 95 weight/weight percent, which includes the endpoints. In another embodiment, the concentration of the antiseptic is in the range of 0.05 to 20 weight/weight percent, which includes the endpoints. In another embodiment, the concentration of the antiseptic is in the range of 0.1 to 10 weight/weight percent, which includes the endpoints.

In another embodiment, the first and second solutions are as described in Table 3.

TABLE 3

| | Mass | Ingredient Conc. |
|---|---|---|
| | (g) | (w/w %) |
| First | | |
| Solution (A) | | |
| Formulation | | |
| Water | 38.26 | 78.52% |
| Surfactant | 4.72 | 9.68% |
| Acid | 4.48 | 9.20% |
| Second | | |
| Solution (B) | | |
| Formulation | | |
| Water | 39.42 | 78.85% |
| Surfactant | 1.62 | 3.23% |
| Sodium | 7.66 | 15.32% |
| Nitrite | | |
| Antiseptic | | |

In this embodiment, the concentration of the antiseptic is in the range of 0.01 to 95 weight/weight percent, which includes the endpoints. In another embodiment, the concentration of the antiseptic is in the range of 0.05 to 20 weight/weight percent, which includes the endpoints. In another embodiment, the concentration of the antiseptic is in the range of 0.1 to 10 weight/weight percent, which includes the endpoints.

In another embodiment, the first and second solutions are as described in Table 4.

TABLE 4

| | Mass | Ingredient Conc. |
|---|---|---|
| | (g) | (w/w %) |
| First | | |
| Solution (A) | | |
| Formulation | | |
| Water | 38.26 | 78.52% |
| Acid | 4.48 | 9.20% |
| Antiseptic | | |
| Second | | |
| Solution (B) | | |
| Formulation | | |
| Water | 39.42 | 78.85% |
| Sodium | 7.66 | 15.32% |
| Nitrite | | |
| Antiseptic | | |

In this embodiment, the concentration of the antiseptic is in the range of 0.01 to 95 weight/weight percent, which includes the endpoints. In another embodiment, the concentration of the antiseptic is in the range of 0.05 to 20 weight/weight percent, which includes the endpoints. In another embodiment, the concentration of the antiseptic is in the range of 0.1 to 10 weight/weight percent, which includes the endpoints.

In another embodiment, the first and second solutions are as described in Table 5.

TABLE 5

| | Mass | Ingredient Conc. |
|---|---|---|
| | (g) | (w/w %) |
| First | | |
| Solution (A) | | |
| Formulation | | |
| Water | 38.26 | 78.52% |
| Acid | 4.48 | 9.20% |
| Antiseptic | | |
| Second | | |
| Solution (B) | | |
| Formulation | | |
| Water | 39.42 | 78.85% |
| Sodium | 7.66 | 15.32% |
| Nitrite | | |

In this embodiment, the concentration of the antiseptic is in the range of 0.01 to 95 weight/weight percent, which includes the endpoints. In another embodiment, the concentration of the antiseptic is in the range of 0.05 to 20 weight/weight percent, which includes the endpoints. In another embodiment, the concentration of the antiseptic is in the range of 0.1 to 10 weight/weight percent, which includes the endpoints.

In another embodiment, the first and second solutions are as described in Table 3.

TABLE 6

| | Mass | Ingredient Conc. |
|---|---|---|
| | (g) | (w/w %) |
| First | | |
| Solution (A) | | |
| Formulation | | |
| Water | 38.26 | 78.52% |
| Acid | 4.48 | 9.20% |
| Second | | |
| Solution (B) | | |
| Formulation | | |
| Water | 39.42 | 78.85% |
| Sodium | 7.66 | 15.32% |
| Nitrite | | |
| Antiseptic | | |

In this embodiment, the concentration of the antiseptic is in the range of 0.01 to 95 weight/weight percent, which includes the endpoints. In another embodiment, the concentration of the antiseptic is in the range of 0.05 to 20 weight/weight percent, which includes the endpoints. In another embodiment, the concentration of the antiseptic is in the range of 0.1 to 10 weight/weight percent, which includes the endpoints.

In another embodiment, the first and second solutions are as described in Table 7.

TABLE 7

| | | | Ingredient | Component |
|---|---|---|---|---|
| | CAS | Mass | Conc. | Concentration |
| | Number | (g) | (w/w %) | (%) |
| First | | | | |
| Solution (A) | | | | |
| Formulation | | | | |
| Deionized | 7732-18-5 | 38.26 | 78.52% | 87.76% |
| Water | | | | |
| CoCo Betaine | 68424-94-2 | 4.72 | 9.68% | 2.90% |
| Citric Acid | 77-92-9 | 4.48 | 9.20% | 9.20% |
| Antiseptic | | | | 0.13% |
| Second | | | | |
| Solution (B) | | | | |
| Formulation | | | | |
| Deionized | 7732-18-5 | 39.42 | 78.85% | 83.58% |
| Water | | | | |
| CoCo Betaine | 68424-94-2 | 1.62 | 3.23% | 0.97% |
| Sodium | 7632-00-0 | 7.66 | 15.32% | 15.32% |
| Nitrite | | | | |
| Antiseptic | | | | 0.13% |

In the embodiments provided in Table 1, Table 2, Table 3, Table 4, Table 5, Table 6, and Table 7, the antiseptic is independently selected from the group consisting of ethanol, isopropanol, hypochlorous acid, benzethonium chloride, benzalkonium chloride (BAC), camphorated metacresol, eucalyptol 0.091%, hexylresorcinol hydrogen peroxide topical solution, iodine tincture, iodine topical solution, menthol, methylbenzethonium chloride, methyl salicylate, phenol, povidone-iodine, and thymol.

In another embodiment, the composition comprises a topical keratolytic selected from the group consisting of podofilox (CAS Reg. No. 518-28-5), salicylic acid, and trichloroacetic acid.

In another embodiment, the composition comprises an emollient selected from the group consisting of salicylic acid/urea, ammonium lactate, urea, vitamin a, d, and e, petrolatum, lanolin, mineral oil, cetyl dimethicone copolyol, cyclomethicone, palm glyceride(s), panthenol (CAS Reg. No. 81-13-0), and aloe vera.

In another embodiment, the composition comprises a topical anti-acne agent selected from the group consisting of adapalene, benzoyl peroxide, clindamycin, dapsone, tretinoin, azelaic acid, tazarotene, salicylic acid, clascoterone, erythromycin, and resorcinol.

In another embodiment, the composition comprises salicylic acid, which may play many roles in a topical composition, including as a topical keratolytic, emollient, or anti-acne agent.

### Resultant Medium

As disclosed herein, when a portion of the first and second solutions are mixed, a resultant mixed medium is produced that contains nitric oxide. In one embodiment, the resultant mixed medium is a liquid, cream, paste, foam, gel, slurry, lotion, emulsion or other medium that is readily applied to the skin.

In one embodiment, the resultant medium is a foam, in which the first and second aqueous solutions are agitated to produce foams, and then the two foams are combined to form the resultant medium. Typically, the first and second aqueous solutions are available in first and second plastic squirt bottles that can be shaken by hand to produce foams or mechanically pumped or otherwise forced through screens to produce foams. The two foams are mixed, typically again by hand, to produce a foam carrier for the in situ generation of nitric oxide due to the reaction of sodium nitrite or potassium nitrite with acid, according to the reaction sequence shown below.

NO₂⁻+H⁺↔HNO₂(pKa3.2 to 3.4) (1)

2HNO₂→N₂O₃+H₂O (2)

N₂O₃→NO+NO₂ (3)

In one embodiment, the resultant medium is a foam, a homogenous liquid (solution) or emulsion.

In another embodiment, the resultant medium is a foam. The use of a foam carrier provides a number of benefits. The foam carrier may effectively keep the nitric oxide contained near the skin, to promote topical treatment of cuts, scrapes, and burns, and to promote the absorption of nitric oxide through the skin. Although not bound by theory, a bubble containing nitric oxide gas may burst near the skin allowing the nitric oxide to be absorbed transdermally. The foam may be placed on the skin and then allowed to collapse as opposed to being rubbed in. The foam carrier may provide a relatively easy process for metering the amount of nitric oxide generated and put in contact with cuts, scrapes, and burns. The foam carrier may also provide a method that uses less media as compared to other media.

In one embodiment, the resultant medium is a topical solution in water. Since the viscosity of the aqueous solution is low, it can be applied as drops, a rinse, a soak, an instillation, as a spray, or by another method such as daubing using a gauze pad or soft cloth soaked in the resultant medium. An emulsion can be similarly applied to skin in need of treatment.

### Skin Conditions and Pathologies Requiring Treatment

As noted above, the combination NO/topical pharmaceutical agent composition disclosed herein can be used on the skin of animals and humans to treat cuts, scrapes, and burns, as well as other conditions and pathologies of the skin. As noted previously, NO is involved in a range of biological processes, including vasodilation, platelet and leukocyte aggregation and adhesion, cell proliferation, endothelial layer permeability, scavenging of superoxide radicals, antimicrobial effects, immunomodulatory effects, and wound repair. The combination NO/topical pharmaceutical agent composition of the present invention is suitable to regulate such processes by the provision of nitric oxide and the transport and delivery of the nitric oxide to the target sites, as well as to provide the treatment benefit of one or more topical pharmaceutical agents without destroying NO. Thus, the combination NO/topical pharmaceutical agent composition of the present invention can be used to treat infections, inflammations, injuries, skin disorders and/or blood vessel disorders in humans and animals. Blood vessel disorders include chronic inflammatory skin diseases, acne, eczema, neurodermatitis, psoriasis, scars, wrinkles, abrasions, burns, trauma, hematoma, androgenetic alopecia (male-pattern hair loss and female-pattern hair loss), blepharitis, Raynaud syndrome, bacterial skin & soft-tissue infections, leg ulcer causes by peripheral vascular disease, chronic venous insufficiency and/or ischemic vasculitis.

### Cuts, Scrapes, Punctures, Skin Infections

In an embodiment, the combination NO/topical pharmaceutical agent composition can be used to disrupt biofilms on the skin, as well as to treat bacterial skin infections and inflammatory skin conditions.

Bacterial skin infections develop when bacteria enter through hair follicles or through small breaks in the skin that results from scrapes, punctures, surgery, burns, sunburn, animal or insect bites, wounds, and pre-existing skin disorders. Bacterial infections of the skin are described, for instance, in the Merck Manual, and include carbuncles, ecthyma, erythrasma, folliculitis, furuncles, impetigo, lymphadenitis, skin abscesses, cellulitis, erysipelas, lymphangitis, necrotizing skin infections, wound infections, staphylococcal scalded skin fever, scarlet fever, toxic shock syndrome. Many types of bacteria can infect the skin. The most common are Staphylococcus aureus (methicillin resistant S. aureus) and Streptococcus.

Biofilm-related infections include chronic wounds caused by diabetes, burns, or surgical procedures. Bacterial infections are common complications of burn wounds, with surface-associated communities of bacteria known as biofilms forming within human burn wounds within 10-24 hours of thermal injury. The presence of biofilms in burns is problematic as biofilms are resistant to antimicrobial agents, thus rendering conventional treatment strategies ineffective, with 75% of extensively burned patients dying as a consequence of severe infection.

Moreover, according to the National Institutes of Health, 65 percent of all hospital-acquired infections are due to bacteria growing as biofilms, and 80 percent of chronic infections are linked to biofilms. Similarly, the Center for Disease Control estimates that hospital-acquired infections account for an estimated 1.7 million infections and 99,000 associated deaths each year in American hospitals alone. The high morbidity and mortality rate is due to biofilms being extremely difficult to control in medical settings.

Conventional therapies have proven inadequate in the treatment of many (if not most) chronic biofilm infections, due to the extraordinary tolerance of biofilms to available antimicrobial agents relative to their planktonic counterparts, and their ability to inhibit healing.

The development of antimicrobial burn creams was considered a major advance in the care of burn wound patients, yet infections of wounds remain the most common cause of morbidity and mortality among the 6.5 million people suffering from wounds in the United States alone, causing over 200,000 deaths annually. The most affected are people suffering from burn wounds, for which almost 61% of deaths are caused by infection. Treatment failure has been primarily attributed to the virulence factors produced by the principal wound pathogens Staphylococcus aureus and Pseudomonas aeruginosa as well as their ability to form biofilms in wounds, which are recalcitrant to antibiotic treatment and the host immune defense.

Successful treatment of biofilm infections will require the development of novel treatment strategies such as the use of the NO containing foam compositions disclosed herein.

Thus, in an embodiment, the combination NO/topical pharmaceutical agent composition can be used to disrupt biofilms by forming a bubble barrier over the biofilm More specifically, the composition can be used to eradicate biofilms. In one embodiment, the biofilms are associated with a microorganism selected from the group consisting of Candida albicans, Coagulase-negative staphylococci, Enterococcus spp., Klebsiella pneumonia, Pseudomonas aeruginosa, and Staphylococcus aureus, Staphylococcus epidermis, Proteus mirabilis, Acinetobacter baumanii, or a combination thereof.

### Inflammatory Disorders of the Skin and Other Conditions

In an embodiment, the combination NO/topical pharmaceutical agent composition can be used to treat inflammatory skin diseases. An estimated 20-25% of the population is affected by chronic, non-communicable inflammatory skin diseases, including atopic dermatitis, psoriasis, urticaria, prurigo nodularis, lichen planus, hidradenitis suppurativa, alopecia areata, vitiligo, urticaria, pemphigus, bullous pemphigoid, mucus membrane pemphigoid, epidermolysis bullosa acquisita, cryopyrin associated periodic disorder, Schnitzier's syndrome, dermatomyositis, and systemic sclerosis. Chronic skin inflammation can also in some rare cases be caused by autoinflammatory diseases, or rheumatic diseases, such as cutaneous lupus erythematosus or dermatomyositis.

The combination NO/topical pharmaceutical agent composition can also be used to treat other conditions, including neuropathy, diabetic neuropathy, and nerve damage.

### Method of Treatment

In one embodiment, the method of using the topical dermatologic composition disclosed herein comprises:
providing the first and second aqueous solutions as recited herein;
dispensing a portion of the first aqueous solution;
dispensing a portion of the second aqueous solution;
mixing the portion of the first aqueous solution and the portion of the second aqueous solution in a manner that produces a resultant mixed medium comprising nitric oxide and an additional pharmaceutically acceptable ingredient; and
applying the resultant medium to the site of the skin condition.

As noted previously, the first and second aqueous solutions are available in first and second plastic squirt bottles that can be shaken by hand or mechanically pumped or otherwise forced to dispense. A portion of the first solution is dispensed and then mixed with a portion of the second solution which is dispensed. The two solutions are mixed, typically again by hand, to produce a resultant carrier for the in situ generation of nitric oxide as described previously. The resultant medium may be allowed to remain on the portion of skin for a period of time so that the resultant nitric oxide can be delivered to the site requiring treatment or an infection.

### Additional Aspects

An additional aspect of the invention is directed to a topical dermatological composition comprising:
a first aqueous solution comprising:
   1-20 by weight percent of at least one protic acid based on the total weight of the first solution;
a second aqueous solution comprising:
   1-35 by weight percent of a nitric oxide precursor based on the total weight of the second solution;
wherein at least one or both of the first and second solutions contain one or more additional topical dermatologic agents; and
wherein a resultant mixed medium containing nitric oxide is produced when the first and second solutions are mixed.

An additional aspect of the invention is directed to a pharmaceutical composition for topical administration comprising: water, nitric oxide, at least one additional topical dermatologic agent, and optionally a surfactant.

An additional aspect of the invention is directed to a pharmaceutical composition for topical administration comprising: water, nitric oxide, at least one additional topical dermatologic agent, and optionally a surfactant.

An additional aspect of the invention is directed to a pharmaceutical composition for topical administration comprising: water, nitric oxide, at least one additional topical dermatologic agent, and optionally a surfactant, wherein the pharmaceutical composition has a pH of 1.5 to 7.0.

### Embodiments

The following non-limiting embodiments illustrate the invention disclosed herein.

Embodiment 1. A topical dermatologic composition comprising:
a first aqueous solution comprising:
   1-20 weight percent of at least one protic acid based on the total weight of the first solution;
   0.05 to 10.2 weight of a surfactant based on the total weight of the first solution;
a second aqueous solution comprising:
   1-35 weight percent of a nitric oxide precursor based on the total weight of the second solution;
   0.05 to 10.2 weight percent of a surfactant based on the total weight of the second solution;
wherein at least one or both of the first and second solutions contain one or more additional topical dermatologic agents;
wherein a resultant mixed medium containing nitric oxide is produced when the first and second solutions are mixed.
   Embodiment 2. The topical dermatologic agent composition of Embodiment 1, wherein the nitric oxide precursor is selected from the group consisting of nitrite, nitrate, diazeniumdiolates (NONOates), L-arginine, L-citrulline, S-nitrosothiols, nitrosamines, nitroglycerin, nitroprusside and NO-metal complexes, or a combination thereof.
   Embodiment 3. The topical dermatologic composition of Embodiment 2, wherein the nitric oxide precursor is selected from the group consisting of sodium nitrite and potassium nitrite, or a combination thereof.
   Embodiment 4. The topical dermatologic composition of Embodiments 1-3 wherein the at least one protic acid is selected from the group consisting of citric acid, lactic acid, salicylic acid, phosphoric acid, ascorbic acid, hydrochloric acid, acetic acid, hyaluronic acid, hypochlorous acid, and honey or a combination thereof.
   Embodiment 5. The topical dermatologic composition of Embodiments 1-4, wherein the at least one protic acid is selected from the group consisting of citric acid, ascorbic acid, hypochlorous acid, and honey, or a combination thereof.
   Embodiment 6. The topical dermatologic composition of Embodiments 1-5, wherein the surfactant is selected from the group consisting of cetyl trimethyl ammonium bromide, cetrimonium bromide; dodecylbenzenesulfonic acid, cetylpyridinium chloride, stearalkonium chloride, polyquaternium-7, coco betaine, cocamidopropyl betaine, lauryl dimethyl ammonium chloride, polyquaternium-10, behentrimonium chloride, and cetrimonium chloride or a combination thereof.
   Embodiment 7. The topical dermatologic composition of Embodiment 6, wherein the surfactant is selected from the group consisting of cocamidopropyl betaine and coco betaine or a combination thereof.
   Embodiment 8. The topical dermatologic composition of Embodiments 1-7, further comprising one or more topical dermatological agents having a therapeutic effect or topical dermatologic excipients, wherein the topical dermatological agent or excipient is selected from the group consisting of topical acne agent, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatoirepical photochemotherapeutics, topical rubefacients, topical steroids, topical steroids with anti-infectives, topical cleansers and topical antiseptics, angiogenesis agents, anorectal therapeutic agents, skin protectants, odor managers, moisture managers, topical enzymes, wound ingredient dressings, topical excipients including one or more of humectants, emulsifiers, preservatives, adhesives, and thickeners.
   Embodiment 9. The topical dermatologic composition of Embodiments 1-8, wherein the topical dermatologic agent is an antiseptic agent.
   Embodiment 10. The topical dermatologic composition of Embodiment 9, wherein the topical dermatologic agent is selected from the group consisting of alcohol (ethanol and isopropanol), benzethonium chloride, benzalkonium chloride (BAC), camphorated metacresol, eucalyptol 0.091%, hexylresorcinol hydrogen peroxide topical solution, iodine tincture, iodine topical solution, menthol, methylbenzethonium chloride, methyl salicylate, phenol, povidone-iodine, hypochlorous acid, and thymol or a combination thereof.
   Embodiment 11. The topical dermatologic composition of Embodiments 1-10, wherein the first and second media are foams or are mixed to produce a foam.
   Embodiment 12. The topical dermatologic composition of Embodiments 1-11, wherein the first solution is an aqueous solution comprising:
1 to 20 percent by weight of the at least one protic acid based on the total weight of the first solution;
0.1 to 10 percent by weight of cocamidopropyl betaine or coco betaine based on the total weight of the first solution; and
0.05 to 0.2 percent by weight of an additional topical dermatologic agent based on the total weight of the first solution.
   Embodiment 13. The topical dermatologic composition of Embodiments 1-12, wherein the second solution is an aqueous solution comprising:
1-35 weight percent of a nitric oxide precursor based on the total weight of the second solution;
0.1 to 10 weight percent of cocamidopropyl betaine or coco-betaine based on the total weight of the second solution; and
0.05 to 0.2 weight percent of an additional topical dermatologic agent based on the total weight of the second solution.
   Embodiment 14. The topical dermatologic composition of Embodiments 1-13, wherein the first solution is an aqueous solution comprising:
1 to 20 percent by weight of the at least one protic acid based on the total weight of the first solution;
0.1 to 10 percent by weight of cocamidopropyl betaine or coco-betaine based on the total weight of the first solution; and
0.05 to 0.2 percent by weight of an additional topical dermatologic agent based on the total weight of the first solution; and
the second solution is an aqueous solution comprising:
   1-35 weight percent of a nitric oxide precursor based on the total weight of the second solution;
   0.1 to 10 weight percent of cocamidopropyl betaine or coco-betaine based on the total weight of the second solution; and
   0.05 to 0.2 weight percent of an additional topical dermatologic agent based on the total weight of the second solution.
      Embodiment 15. A method for treating a dermatologic condition with a topical dermatologic composition in a subject in need of such treatment, comprising:
providing the first and second aqueous solutions as recited in Embodiments 1-14;
dispensing a portion of the first aqueous solution;
dispensing a portion of the second aqueous solution;
mixing the portion of the first aqueous solution and the portion of the second aqueous solution in a manner that produces a resultant mixed medium comprising nitric oxide; and
applying the resultant medium to the site of the skin condition.
   Embodiment 16. A method for treating a surface of a subject in need of such treatment, comprising:
providing the first and second solutions as recited in Embodiments 1-14;
dispensing a portion of the first solution;
dispensing a portion of the second solution;
mixing the portion of the first solution and the portion of the second solution in a manner that produces a resultant medium that includes a plurality of bubbles, wherein the bubbles contain nitric oxide and the bubble film is comprised in part of the additive ingredient;
applying the resultant medium to the skin;
allowing a first portion of the plurality of bubbles in the resultant medium to burst next to the skin, releasing nitric oxide to the application site;
allowing the residual bubble film comprised in part of the additive ingredient to come into contact with the skin, and
treating the application site with nitric oxide from the bubbles and the additive ingredient from the bubble film.
   Embodiment 17. A topical dermatological composition comprising:
a first aqueous solution comprising:
   1-20 by weight percent of at least one protic acid based on the total weight of the first solution;
a second aqueous solution comprising:
   1-35 by weight percent of a nitric oxide precursor based on the total weight of the second solution;
wherein at least one or both of the first and second solutions contain one or more additional topical dermatologic agents; and
wherein a resultant mixed medium containing nitric oxide is produced when the first and second solutions are mixed.
   Embodiment 18. The topical dermatologic agent composition of Embodiment 17, wherein the nitric oxide precursor is selected from the group consisting of nitrite, nitrate, diazeniumdiolates (NONOates), S-nitrosothiols, nitrosamines, nitroglycerin, nitroprusside and NO-metal complexes, or a combination thereof.
   Embodiment 19. The topical dermatologic composition of Embodiment 18, wherein the nitric oxide precursor is selected from the group consisting of sodium nitrite and potassium nitrite, or a combination thereof
   Embodiment 20. The topical dermatologic composition of Embodiments 17-19 wherein the at least one protic acid is selected from the group consisting of citric acid, lactic acid, salicylic acid, phosphoric acid, ascorbic acid, hydrochloric acid, acetic acid, hyaluronic acid, hypochlorous acid, and honey or a combination thereof.
   Embodiment 21. The topical dermatologic composition of Embodiments 17-20, wherein the at least one protic acid is selected from the group consisting of citric acid, ascorbic acid, hypochlorous acid, and honey, or a combination thereof.
   Embodiment 22. The topical dermatologic composition of Embodiments 17-21, further comprising one or more topical dermatological agents having a therapeutic effect or topical dermatologic excipients, wherein the topical dermatological agent or excipient is selected from the group consisting of topical acne agent, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacients, topical steroids, topical steroids with anti-infectives, topical cleansers and topical antiseptics, angiogenesis agents, anorectal therapeutic agents, skin protectants, odor managers, moisture managers, topical enzymes, wound ingredient dressings, topical excipients including one or more of humectants, emulsifiers, preservatives, adhesives, and thickeners.
   Embodiment 23. The topical dermatologic composition of Embodiments 17-22, wherein the topical dermatologic agent is an antiseptic agent.
   Embodiment 24. The topical dermatologic composition of Embodiment 23, wherein the topical dermatologic agent is selected from the group consisting of alcohol (ethanol and isopropanol), benzethonium chloride, benzalkonium chloride (BAC), camphorated metacresol, eucalyptol 0.091%, hexylresorcinol hydrogen peroxide topical solution, iodine tincture, iodine topical solution, menthol, methylbenzethonium chloride, methyl salicylate, phenol, povidone-iodine, hypochlorous acid, and thymol or a combination thereof.
   Embodiment 25. The topical dermatologic composition of Embodiments 17-24, wherein the first solution is an aqueous solution comprising:
1 to 20 percent by weight of the at least one protic acid based on the total weight of the first solution; and
0.05 to 0.2 percent by weight of an additional topical dermatologic agent based on the total weight of the first solution.
   Embodiment 26. The topical dermatologic composition of Embodiments 17-24, wherein the second solution is an aqueous solution comprising:
1-35 weight percent of a nitric oxide precursor based on the total weight of the second solution; and
0.05 to 0.2 weight percent of an additional topical dermatologic agent based on the total weight of the second solution.
   Embodiment 27. The topical dermatologic composition of Embodiments 17-24, wherein the first solution is an aqueous solution comprising:
1 to 20 percent by weight of the at least one protic acid based on the total weight of the first solution; and
0.05 to 0.2 percent by weight of an additional topical dermatologic agent based on the total weight of the first solution; and
the second solution is an aqueous solution comprising:
   1-35 weight percent of a nitric oxide precursor based on the total weight of the second solution; and
   0.05 to 0.2 weight percent of an additional topical dermatologic agent based on the total weight of the second solution.
      Embodiment 28. A method for treating a dermatologic condition with a topical dermatologic composition in a subject in need of such treatment, comprising:
providing the first and second aqueous solutions as recited in Embodiments 17-27;
dispensing a portion of the first aqueous solution;
dispensing a portion of the second aqueous solution;
mixing the portion of the first aqueous solution and the portion of the second aqueous solution in a manner that produces a resultant mixed medium comprising nitric oxide; and
applying the resultant medium to the site of the skin condition.
   Embodiment 29. A method for treating a surface of a subject in need of such treatment, comprising:
providing the first and second solutions as recited in Embodiments 17-27;
dispensing a portion of the first solution;
dispensing a portion of the second solution;
mixing the portion of the first solution and the portion of the second solution in a manner that produces a resultant medium that includes a plurality of bubbles, wherein the bubbles contain nitric oxide and the bubble film is comprised in part of the additive ingredient;
applying the resultant medium to the skin;
allowing a first portion of the plurality of bubbles in the resultant medium to burst next to the skin, releasing nitric oxide to the application site;
allowing the residual bubble film comprised in part of the additive ingredient to come into contact with the skin, and
treating the application site with nitric oxide from the bubbles and the additive ingredient from the bubble film.
   Embodiment 30. A pharmaceutical composition for topical administration comprising: water, nitric oxide, at least one additional topical dermatologic agent, and optionally a surfactant.
   Embodiment 31. A pharmaceutical composition for topical administration comprising: water, nitric oxide, at least one additional topical dermatologic agent, and optionally a surfactant, wherein the pharmaceutical composition has a pH of 5.0 to 7.0.

### EXAMPLES

### Example 1: Topical NO/Antiseptic Composition

A two-part topical dermatological composition for treating skin conditions that contained benzalkonium chloride (BAC) was tested to determine the amount of nitric oxide generated in situ as compared to a two-part topical dermatological composition that did not contain benzalkonium chloride. The components of the two-part topical dermatological composition are summarized in Table 1A.

TABLE 1A

| | CAS | | Mass | Ingredient | | Component |
|---|---|---|---|---|---|---|
| | Number | Description | (g) | Conc. (w/w %) | Component | Concentration (%) |
| First (A) Solution | | | | | | |
| Deionized Water | 7732-18-5 | Deionized water, Conductivity ≤4.3 μS/cm | 38.26 | 78.52% | DI Water | 87.76% |
| CoCo Betaine | 68424-94-2 | 30% actives solution in purified water (BASF | 4.72 | 9.68% | Coco Betaine | 2.90% |
| | | Dehyton AB-30) | | | | |
| Citric Acid | 77-92-9 | Powder, UPC grade | 4.48 | 9.20% | Citric Acid | 9.20% |
| Benzalkonium Chloride | 8001-54-5 | 5% actives solution in purified water | 1.27 | 2.60% | BZK | 0.13% |
| Second (B) Formulation | | | | | | |
| Deionized Water | 7732-18-5 | Deionized water, Conductivity ≤4.3 μS/cm | 39.42 | 78.85% | DI Water | 83.58% |
| CoCo Betaine | 68424-94-2 | 30% actives solution in purified water (BASF | 1.62 | 3.23% | Coco Betaine | 0.97% |
| | | Dehyton AB-30) | | | | |
| Sodium Nitrite | 7632-00-0 | Powder, Food grade, no flow enhancers or | 7.66 | 15.32% | Citric Acid | 15.32% |
| | | anti-caking agents | | | | |
| Benzalkonium Chloride | 8001-54-5 | 5% actives solution in purified water | 1.30 | 2.60% | BZK | 0.13% |

The initial components shown in Table 1A were used to prepare two aqueous solutions, which were utilized in the testing process.

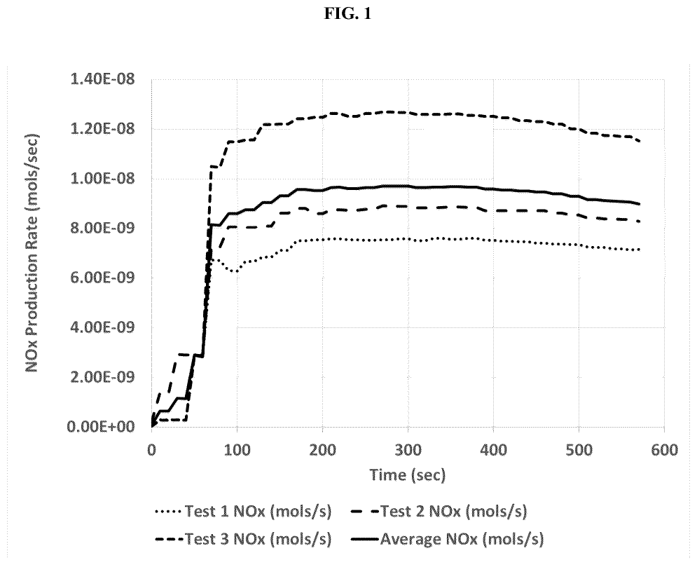

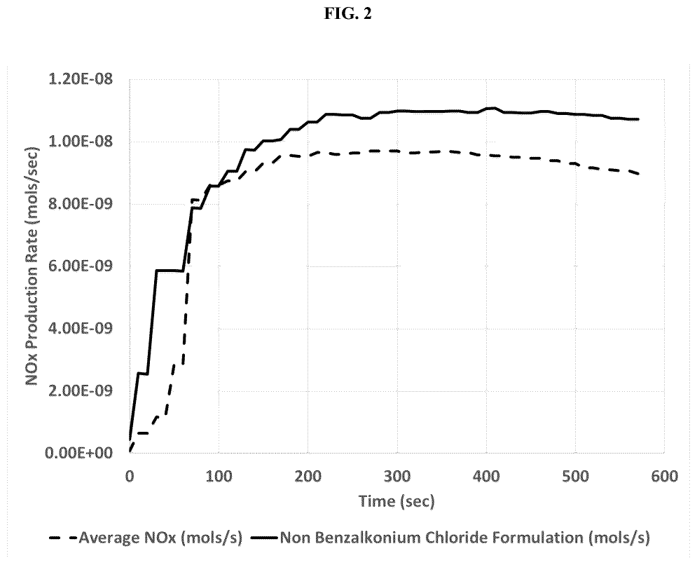

Methods. Solutions A and B were agitated by vigorous shaking until they produced foams. Then, two pumps of foam A (from Solution A) and foam B (from Solution B) were added to a weigh boat, and then the resulting mixture was thoroughly mixed with a gloved finger for five seconds. The resulting mixture was then placed in the sample chamber for measurement using a Thermoscientific 42iQLS Gas Analyzer. The measured concentration of the NOx produced was recorded every ten seconds. Using the Ideal Gas Law, the mass in mols of NOx was calculated. It is this calculated mass production rate that is shown in FIGS. 1 and 2.

Results. The production profiles for three test examples are illustrated in FIG. 1 against a standard that does not contain benzalkonium chloride (FIG. 1). The average production profile of the three BAC tests from FIG. 1 is also provided in FIG. 2 for comparison.

Conclusion. The BAC solutions were found to produce an equivalent amount of NO when compared to the standard that did not contain BAC, suggesting that other antiseptic additives can be employed without deleterious effect to the product of the foam carrier or the in situ generation of nitric oxide.

### Example 2: Topical NO/Salicylic Acid Foam Composition

Tests were conducted to measure the NOx (NO and NO₂) production rate of a topical NO/Salicylic Acid composition foam product. As noted previously, salicylic acid plays many roles in a topical composition, including as a topical keratolytic, emollient, or anti-acne agent.

The first (A) solution was prepared as described in Example 1, except BAC was not included. The second (B) solution was prepared by dissolving salicylic acid in a stoichiometric amount of sodium hydroxide dissolved in water. Once all of the salicylic acid was dissolved, sodium nitrite and coco betaine were added. The first (A) and second (B) solutions were then mixed. A foam was produced upon the mixing/priming of the first (A) solution and the second (B) solution.

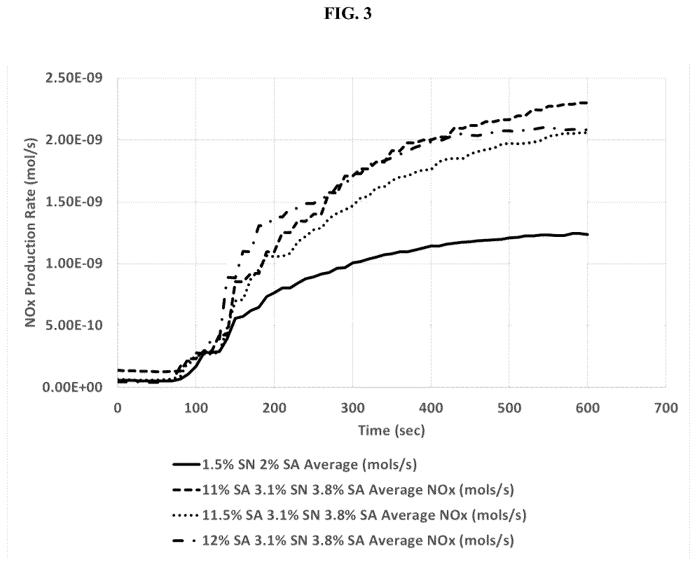

As FIG. 3 indicates, the amount of NOx produced by the solutions was almost as much as predicted. Although not bound by theory, the slight reduction in NOx production may be due to sodium hydroxide competing with sodium nitrite for protons.

### Example 3: Topical NO/Ascorbic Acid Composition

Three solutions of ascorbic acid (0.4 M, 0.5 M, and 0.6 M) were prepared and used as first (A) solutions. The second (B) solution was as provided in Example 1 except that BAC was not included. The solutions were poured into small foam dispensers. One squirt of foam from each of the first (A) solution and the second (B) solution was combined in a weight boat and NOx formation was measured.

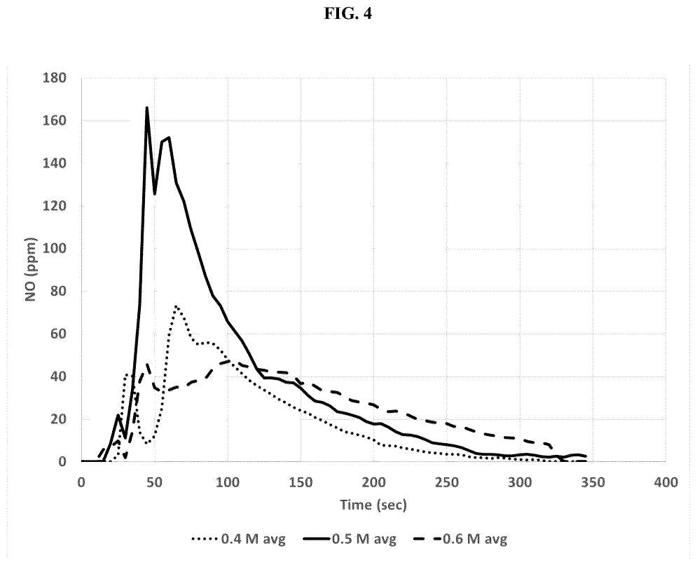

FIG. 4 indicates that use of ascorbic acid leads to lower levels of production of NOx than citric acid. Although not bound by theory, ascorbic acid is not as acidic as citric acid, which leads to fewer protons to react with sodium nitrite.

### Example 4: Topical NO/Buffered Hypochlorous Acid Composition

Buffered (pH balanced) hypochlorous acid is a component of commercial antiseptic washes that are used in wound care products. In this experiment, a buffered hypochlorous acid skin antiseptic solution served as the first (A) solution, which was mixed with the second (B) solution in Experiment 1 (except that BAC was not present).

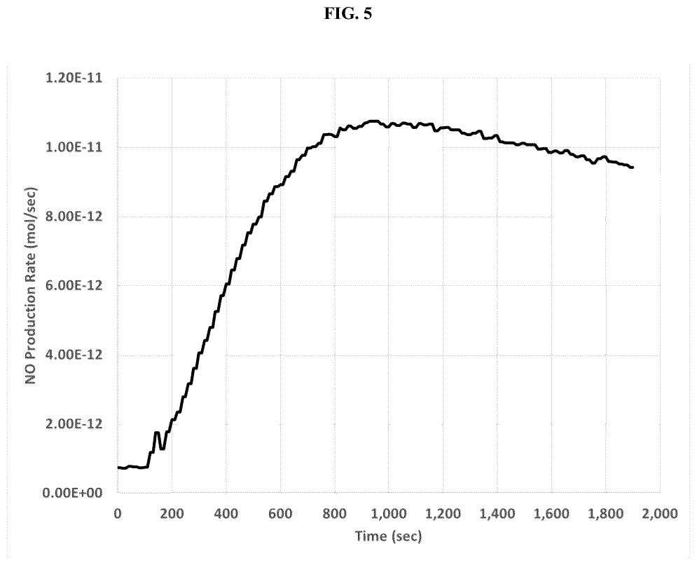

FIG. 5 shows that NO was produced, although the pH of the solution is not as low as first (A) solutions containing citric acid.

### Example 5: Topical NO/Lidocaine Composition

Lidocaine, along with pramoxine, phenol, benzocaine, dibucaine, xylocaine, tetracaine, prilocaine, benzocaine, wintergreen (CAS Reg. No. 68917-75-9), and dyclonine, are topical anesthetics. In this experiment, a 4 percent solution of lidocaine HCl (pH=4.16) in water was used as the first (A) solution. NO was produced when the 4 percent aqueous lidocaine HCl solution was mixed with the second (B) solution described in Experiment 1 (except BAC was not present). NO was also produced when a mixture of 4 percent aqueous lidocaine HCl and the first (A) solution (not containing BAC) solution was mixed with the second (B) solution described in Experiment 1 (not containing BAC).

### Objective of Test and Tests Performed

The objective of the testing was to show that a 4% lidocaine HCl solution (which is twice the concentration allowed by the Food & Drug Administration (FDA) in topical dermatologic products could be mixed into the first (A) solution in a stable manner. Another objective was to measure the NO produced from mixing the lidocaine HCl solution with the second (B) (not including BAC) solution. The final objective was to show that NO would be released from standard first (A) and second (B) solutions (not including BAC) reaction without interference from the added lidocaine HCl.

### Test Methods

A 4% w/w solution of lidocaine in deionized water was prepared. The pH of the solution was measured using a conventional calibrated lab pH meter.

For the NO release testing, one mL of each solution was dispensed into a weight boat using the pipetter. A new pipette tip was used for each aliquot. For NO release measurements, the weigh boat was then placed into the sampling bin of a Thermoscientific 42iQLS Gas Analyzer, and the lid placed on top. The analyzer began measuring NOx released from the mixed liquid in the weigh boat as indicated by the rise of both the NO and NO₂ measurements on the screen of the analyzer, a 10-minute timer was started.

Once the datalogger had collected 70 time points (700 seconds) of data:
1. the data logging program was halted
2. The weigh boat was removed from the chamber and disposed of in a separate room
3. A fan was used to blow fresh air into the testing chamber for 10 seconds
4. The next test was conducted when the analyzer measured less than 5 ppm total NOx

### Results

No reaction was observed when the lidocaine HCl solution was mixed with the standard first (A) solution A (not including BAC). The monitoring time was three minutes.

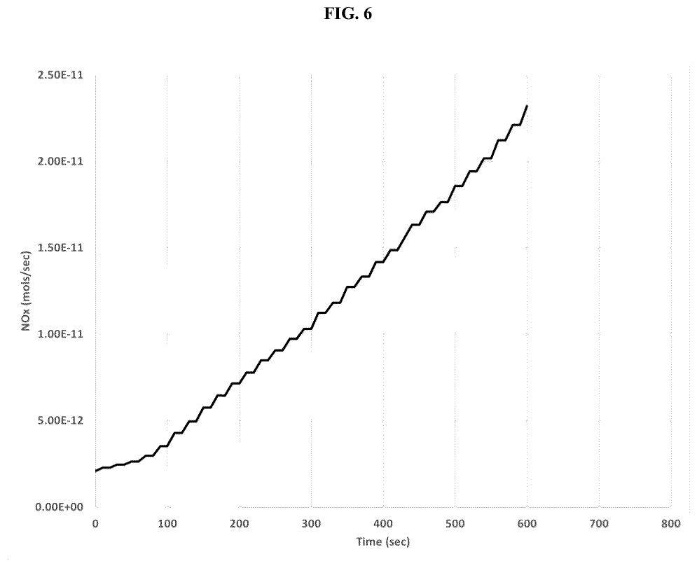

The profile of NOx release when the lidocaine HCl solution was mixed with the standard second (B) solution (not including BAC) is depicted in FIG. 6. Only a small amount of NOx was released.

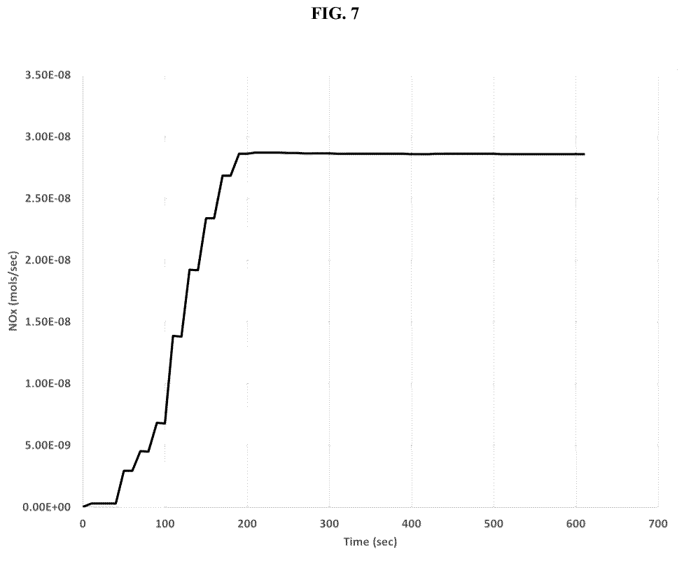

The profile of NOx release was then measured when the lidocaine HCl solution was mixed with standard second (B) solution (not including BAC) and the standard first (A) solution (not including BAC). The results are depicted in FIG. 7. As FIG. 7 indicates, a large amount of NOx was released.

### CONCLUSION

Lidocaine HCl can be incorporated into existing NO-generating compositions as described herein.

### Example 6: Topical NO/Manuka Honey Composition

Honey, particularly Manuka honey, can be used as a topical dermatologic agent. Manuka honey contains the enzyme glucose oxidase that produces gluconic acid and small amounts of hydrogen peroxide when in contact with wound surfaces. Honey has an acidic pH of 4.4, which helps wound healing by changing the pH of the wound from basic (preferred by bacteria) to acidic.

In this experiment, the standard first (A) solution was replaced by Manuka honey, and NOx production was measured upon mixing with the standard second (B) solution (not including BAC) was measured using a Thermoscientific 42iQLS Gas Analyzer.

### Test Method

About 1 mL of Manuka honey was mixed with 1 mL of standard second (B) solution (not including BAC).

### Results and Conclusion

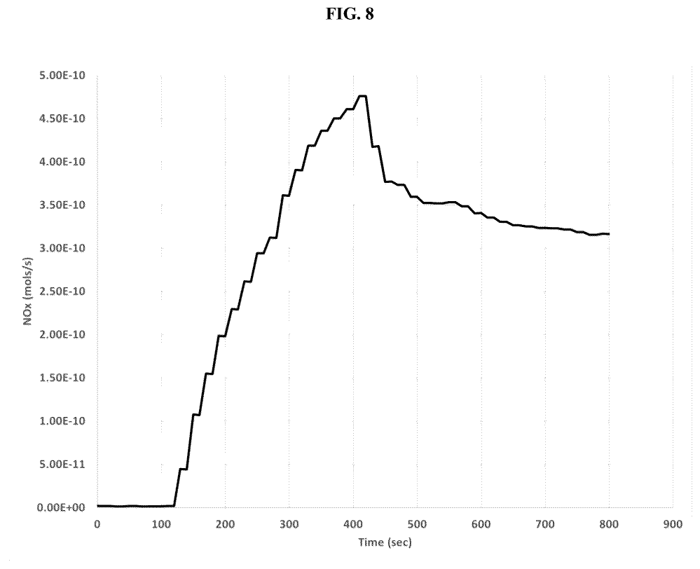

As indicated by FIG. 8, Manuka honey is acidic enough to produce NO from a sodium nitrite solution.

### Example 8: Topical NO/Aloe Vera Composition

According to the NIH/National Center for Complementary and Integrative Health, Aloe is a cactus-like plant that grows in hot, dry climates that has been used topically to treat skin wounds and skin conditions such as acne, rashes such as lichen planus, burns, and sunburns.

In this experiment, the standard first (A) solution was replaced by a 10 percent solution of Aloe Vera gel in water. NOx production was measured upon mixing with the standard second (B) solution (not including BAC) using a Thermoscientific 42iQLS Gas Analyzer. No NOx was measured, indicating that an aqueous dilute solution of Aloe Vera was not a strong enough acid to generate NOx. As a result, a second (B) solution containing Aloe Vera would be stable, and would not lead to decomposition of sodium nitrite.

### Example 8: Use of Table 1A Composition to Treat Wounds

The topical dermatologic composition depicted in Table 1A was used to treat human patient subjects having skin wounds. The patient subjects (n=50) were 69-99 years old (median 85 years old) with pressure wounds (35), skin tears (9), arterial ulcers (2) or other (4) wounds, and had various pre-existing conditions, including diabetes mellitus, cardiovascular disease, peripheral vascular disease. Wounds were generally at the sacrum, coccyx, buttock, hip, leg, toe, or another location. Skin tears were generally at the arm, hand or leg. Arterial ulcers were generally at the leg or heel. Other wounds included redness or other trauma at the arm or leg.

The formulation in Table 1A was most commonly administered every other day (Monday, Wednesday, Friday; 81 percent). Other administration at a dose of 1 pump from a spray bottle/cm². Treatment times ranged from 1 to 34 days, with a mean duration of 9 days.

Wound healing was observed after a mean treatment time of 14 days for ninety eight percent (n=50), of the patient subjects who completed the treatment, as further described in Table 1B.

TABLE 1B

| | | Pressure | | |
|---|---|---|---|---|
| Wound | Skin Tears | Injuries | Arterial Ulcers | Other |
| Treatment Days | 11 | 14 | 22 | 4 |
| Healing (%) | 100 | 86 | 50 | 50 |
| Number | 8 | 35 | 2 | 4 |

Wound healing outcomes by pressure wound stage and time are provided in Table 1C. Stage 1 wounds are characterized as skin that skin is still intact with a localized area of redness that does not turn white when pressure is applied (also known as non-blanchable erythema). Stage 1 does not include purple or maroon discoloration; these may indicate deep tissue pressure injury. Stage 2 wounds are characterized as wounds where there is a partial-thickness loss of skin with exposed dermis. The wound bed is pink or red, moist, and may also present as an intact or ruptured serum-filled blister. Fat and deeper tissues are not visible. Stage 3 wounds involve full-thickness loss of skin, where fat tissue and granulation tissue is visible. Stage 4 wounds full-thickness skin and tissue loss has occurred, with exposed fascia, muscle, tendon, ligament, cartilage, or bone. Unstageable wounds occur when the extent of tissue damage within the ulcer cannot be ascertained. Deep Tissue Pressure Injuries (DTPI) occur in tissue that has been subjected to pressures that exceed the tolerance level of muscle tissue. The muscle cell is deformed and irreversibly injured from the pressure and the membrane of the muscle cell is fractured.

TABLE 1C

| | Stage | Stage | Stage | Stage | | |
|---|---|---|---|---|---|---|
| Wound Stage | 1 | 2 | 3 | 4 | Unstageable | DTPI |
| Treatment Days | 15 | 14 | 18 | 22 | 14 | 14 |
| Healing (%) | 88 | 90 | 100 | 0 | 100 | 91 |
| Number | 8 | 10 | 2 | 1 | 2 | 11 |

It is known that once injuries such as listed in Table 1B form in the elderly in particular, it can take days, months, or even years to heal. It can also become infected, causing fever and chills. For example, an infected pressure injury can take a very long time to clear up, or it can even turn into a chronic wound that does not heal. In severe cases, such injuries can affect the underlying bone (osteomyelitis).

In short, the Table 1A pharmaceutical composition successfully healed the wounds of the majority of patients that participated in the study in less than a month. The results are remarkable, considering the short healing times and the severity of the skin wounds observed in the patient subjects. The results provide strong support for using this formulation for the beneficial treatment of human patients as well as animal of all ages and skin wound types.

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

1. A topical dermatologic composition comprising:
a first aqueous solution comprising:
1-20 weight percent of at least one protic acid based on the total weight of the first solution;
0.05 to 10.2 weight of a surfactant based on the total weight of the first solution;
a second aqueous solution comprising:
1-35 weight percent of a nitric oxide precursor based on the total weight of the second solution;
0.05 to 10.2 weight percent of a surfactant based on the total weight of the second solution;
wherein at least one or both of the first and second solutions contain one or more additional topical dermatologic agents;
wherein a resultant mixed medium containing nitric oxide is produced when the first and second solutions are mixed.

2. The topical dermatologic agent composition of claim 1, wherein the nitric oxide precursor is selected from the group consisting of nitrite, nitrate, diazeniumdiolates (NONOates), L-arginine, L-citrulline, S-nitrosothiols, nitrosamines, nitroglycerin, nitroprusside and NO-metal complexes, or a combination thereof.

3. The topical dermatologic composition of claim 2, wherein the nitric oxide precursor is selected from the group consisting of sodium nitrite and potassium nitrite, or a combination thereof.

4. The topical dermatologic composition of claims 1-3 wherein the at least one protic acid is selected from the group consisting of citric acid, lactic acid, salicylic acid, phosphoric acid, ascorbic acid, hydrochloric acid, acetic acid, hyaluronic acid, hypochlorous acid, and honey or a combination thereof.

5. The topical dermatologic composition of claims 1-4, wherein the at least one protic acid is selected from the group consisting of citric acid, ascorbic acid, hypochlorous acid, and honey, or a combination thereof.

6. The topical dermatologic composition of claims 1-5, wherein the surfactant is selected from the group consisting of cetyl trimethyl ammonium bromide, cetrimonium bromide; dodecylbenzenesulfonic acid, cetylpyridinium chloride, stearalkonium chloride, polyquaternium-7, coco betaine, cocamidopropyl betaine, lauryl dimethyl ammonium chloride, polyquaternium-10, behentrimonium chloride, and cetrimonium chloride or a combination thereof.

7. The topical dermatologic composition of claim 6, wherein the surfactant is selected from the group consisting of cocamidopropyl betaine and coco betaine or a combination thereof.

8. The topical dermatologic composition of claims 1-7, further comprising one or more topical dermatological agents having a therapeutic effect or topical dermatologic excipients, wherein the topical dermatological agent or excipient is selected from the group consisting of topical acne agent, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacients, topical steroids, topical steroids with anti-infectives, topical cleansers and topical antiseptics, angiogenesis agents, anorectal therapeutic agents, skin protectants, odor managers, moisture managers, topical enzymes, wound ingredient dressings, topical excipients including one or more of humectants, emulsifiers, preservatives, adhesives, and thickeners, or a combination thereof.

9. The topical dermatologic composition of claims 1-8, wherein the topical dermatologic agent is an antiseptic agent.

10. The topical dermatologic composition of claim 9, wherein the topical dermatologic agent is selected from the group consisting of alcohol (ethanol and isopropanol), benzethonium chloride, benzalkonium chloride (BAC), camphorated metacresol, eucalyptol 0.091%, hexylresorcinol hydrogen peroxide topical solution, iodine tincture, iodine topical solution, menthol, methylbenzethonium chloride, methyl salicylate, phenol, povidone-iodine, hypochlorous acid, and thymol or a combination thereof.

11. The topical dermatologic composition of claims 1-10, wherein the first and second media are foams or are mixed to produce a foam.

12. The topical dermatologic composition of claims 1-11, wherein the first solution is an aqueous solution comprising:
1 to 20 percent by weight of the at least one protic acid based on the total weight of the first solution;
0.1 to 10 percent by weight of cocamidopropyl betaine or coco betaine based on the total weight of the first solution; and
0.05 to 0.2 percent by weight of an additional topical dermatologic agent based on the total weight of the first solution.

13. The topical dermatologic composition of claims 1-12, wherein the second solution is an aqueous solution comprising:
1-35 weight percent of a nitric oxide precursor based on the total weight of the second solution;
0.1 to 10 weight percent of cocamidopropyl betaine or coco-betaine based on the total weight of the second solution; and
0.05 to 0.2 weight percent of an additional topical dermatologic agent based on the total weight of the second solution.

14. The topical dermatologic composition of claims 1-13, wherein the first solution is an aqueous solution comprising:
1 to 20 percent by weight of the at least one protic acid based on the total weight of the first solution;
0.1 to 10 percent by weight of cocamidopropyl betaine or coco-betaine based on the total weight of the first solution; and
0.05 to 0.2 percent by weight of an additional topical dermatologic agent based on the total weight of the first solution; and
the second solution is an aqueous solution comprising:
1-35 weight percent of a nitric oxide precursor based on the total weight of the second solution;
0.1 to 10 weight percent of cocamidopropyl betaine or coco-betaine based on the total weight of the second solution; and
0.05 to 0.2 weight percent of an additional topical dermatologic agent based on the total weight of the second solution.

15. A method for treating a dermatologic condition with a topical dermatologic composition in a subject in need of such treatment, comprising:
providing the first and second aqueous solutions as recited in claims 1-14;
dispensing a portion of the first aqueous solution;
dispensing a portion of the second aqueous solution;
mixing the portion of the first aqueous solution and the portion of the second aqueous solution in a manner that produces a resultant mixed medium comprising nitric oxide; and
applying the resultant medium to the site of the skin condition.

16. A method for treating a surface of a subject in need of such treatment, comprising:
providing the first and second solutions as recited in claims 1-14;
dispensing a portion of the first solution;
dispensing a portion of the second solution;
mixing the portion of the first solution and the portion of the second solution in a manner that produces a resultant medium that includes a plurality of bubbles, wherein the bubbles contain nitric oxide and the bubble film is comprised in part of the additive ingredient;
applying the resultant medium to the skin;
allowing a first portion of the plurality of bubbles in the resultant medium to burst next to the skin, releasing nitric oxide to the application site;
allowing the residual bubble film comprised in part of the additive ingredient to come into contact with the skin, and
treating the application site with nitric oxide from the bubbles and the additive ingredient from the bubble film.

17. A topical dermatological composition comprising:
a first aqueous solution comprising:
1-20 by weight percent of at least one protic acid based on the total weight of the first solution;
a second aqueous solution comprising:
1-35 by weight percent of a nitric oxide precursor based on the total weight of the second solution;
wherein at least one or both of the first and second solutions contain one or more additional topical dermatologic agents; and
wherein a resultant mixed medium containing nitric oxide is produced when the first and second solutions are mixed.

18. The topical dermatologic agent composition of claim 17, wherein the nitric oxide precursor is selected from the group consisting of nitrite, nitrate, diazeniumdiolates (NONOates), S-nitrosothiols, nitrosamines, nitroglycerin, nitroprusside and NO-metal complexes, or a combination thereof.

19. The topical dermatologic composition of claim 18, wherein the nitric oxide precursor is selected from the group consisting of sodium nitrite and potassium nitrite, or a combination thereof.

20. The topical dermatologic composition of claims 17-19 wherein the at least one protic acid is selected from the group consisting of citric acid, lactic acid, salicylic acid, phosphoric acid, ascorbic acid, hydrochloric acid, acetic acid, hyaluronic acid, hypochlorous acid, and honey or a combination thereof.

21. The topical dermatologic composition of claims 17-20, wherein the at least one protic acid is selected from the group consisting of citric acid, ascorbic acid, hypochlorous acid, and honey, or a combination thereof.

22. The topical dermatologic composition of claims 17-21, further comprising one or more topical dermatological agents having a therapeutic effect or topical dermatologic excipients, wherein the topical dermatological agent or excipient is selected from the group consisting of topical acne agent, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacients, topical steroids, topical steroids with anti-infectives, topical cleansers and topical antiseptics, angiogenesis agents, anorectal therapeutic agents, skin protectants, odor managers, moisture managers, topical enzymes, wound ingredient dressings, topical excipients including one or more of humectants, emulsifiers, preservatives, adhesives, and thickeners or combinations thereof.

23. The topical dermatologic composition of claims 17-22, wherein the topical dermatologic agent is an antiseptic agent.

24. The topical dermatologic composition of claim 23, wherein the topical dermatologic agent is selected from the group consisting of alcohol (ethanol and isopropanol), benzethonium chloride, benzalkonium chloride (BAC), camphorated metacresol, eucalyptol 0.091%, hexylresorcinol hydrogen peroxide topical solution, iodine tincture, iodine topical solution, menthol, methylbenzethonium chloride, methyl salicylate, phenol, povidone-iodine, hypochlorous acid, and thymol or a combination thereof.

25. The topical dermatologic composition of claims 17-24, wherein the first solution is an aqueous solution comprising:
1 to 20 percent by weight of the at least one protic acid based on the total weight of the first solution; and
0.05 to 0.2 percent by weight of an additional topical dermatologic agent based on the total weight of the first solution.

26. The topical dermatologic composition of claims 17-24, wherein the second solution is an aqueous solution comprising:
1-35 weight percent of a nitric oxide precursor based on the total weight of the second solution; and
0.05 to 0.2 weight percent of an additional topical dermatologic agent based on the total weight of the second solution.

27. The topical dermatologic composition of claims 17-24, wherein the first solution is an aqueous solution comprising:
1 to 20 percent by weight of the at least one protic acid based on the total weight of the first solution; and
0.05 to 0.2 percent by weight of an additional topical dermatologic agent based on the total weight of the first solution; and
the second solution is an aqueous solution comprising:
1-35 weight percent of a nitric oxide precursor based on the total weight of the second solution; and
0.05 to 0.2 weight percent of an additional topical dermatologic agent based on the total weight of the second solution.

28. A method for treating a dermatologic condition with a topical dermatologic composition in a subject in need of such treatment, comprising:
providing the first and second aqueous solutions as recited in claims 17-27;
dispensing a portion of the first aqueous solution;
dispensing a portion of the second aqueous solution;
mixing the portion of the first aqueous solution and the portion of the second aqueous solution in a manner that produces a resultant mixed medium comprising nitric oxide; and
applying the resultant medium to the site of the skin condition.

29. A method for treating a surface of a subject in need of such treatment, comprising:
providing the first and second solutions as recited in claims 17-27;
dispensing a portion of the first solution;
dispensing a portion of the second solution;
mixing the portion of the first solution and the portion of the second solution in a manner that produces a resultant medium that includes a plurality of bubbles, wherein the bubbles contain nitric oxide and the bubble film is comprised in part of the additive ingredient;
applying the resultant medium to the skin;
allowing a first portion of the plurality of bubbles in the resultant medium to burst next to the skin, releasing nitric oxide to the application site;
allowing the residual bubble film comprised in part of the additive ingredient to come into contact with the skin, and
treating the application site with nitric oxide from the bubbles and the additive ingredient from the bubble film.

30. A pharmaceutical composition for topical administration comprising: water, nitric oxide, at least one additional topical dermatologic agent, and optionally a surfactant.

31. A pharmaceutical composition for topical administration comprising: water, nitric oxide, at least one additional topical dermatologic agent, and optionally a surfactant, wherein the pharmaceutical composition has a pH of 5.0 to 7.0.
